(19) United States
(12) Patent Application Publication
(10) Pub. No.: US 2024/0021311 A1
(43) Pub. Date: Jan. 18, 2024

(54) Metadata-Based Diagnosis and Management System for Cardiovascular and Cerebrovascular Diseases

(71) Applicant: Serv Medical Pte. Ltd., Singapore (SG)

(72) Inventors: Chun-Chieh HSU, Beijing (CN); Yupei MA, Beijing (CN); Jing ZHANG, Beijing (CN); Bo ZHOU, Beijing (CN); Rongrong JI, Beijing (CN)

(21) Appl. No.: 18/350,726

(22) Filed: Jul. 11, 2023

(30) Foreign Application Priority Data
Jul. 12, 2022 (CN) 202210815840.8

(51) Int. Cl. G16H 50/20; G16H 70/60; G16H 10/60

(52) U.S. Cl. CPC G16H 50/20; G16H 70/60; G16H 10/60; G16H 50/30

(57) ABSTRACT

The present invention discloses a metadata-based diagnosis and management system for cardiovascular and cerebrovascular diseases, which belongs to the field of cardiovascular and cerebrovascular systems and is used to resolve a problem that a rapid diagnosis and a preliminary diagnosis of cardiovascular and cerebrovascular diseases cannot be quickly implemented at community hospitals, clinics, and other places with limited medical facilities. The system includes a physical status monitoring module, a preliminary diagnosis module, an electrocardiogram comparison module, and a model building module.

### CROSS-REFERENCE TO RELATED APPLICATION

This application claims priority to, and the benefit of, Chinese Patent Application No. 202210815840.8 filed on 12 Jul. 2022. The entire contents of the foregoing application are hereby incorporated by reference for all purposes.

### TECHNICAL FIELD

The present invention belongs to the field of cardiovascular and cerebrovascular systems, and relates to disease diagnosis and management technologies, and specifically, a metadata-based diagnosis and management system for cardiovascular and cerebrovascular diseases.

### BACKGROUND OF THE INVENTION

Cardiovascular and cerebrovascular accidents are common diseases that seriously threaten the health of human beings, especially middle-aged and elderly people over 50 years old. Even with the use of advanced and comprehensive therapies, more than 50% of survivors of cerebrovascular accidents cannot take care of themselves.

In the prior art, diagnosis of cardiovascular and cerebrovascular diseases mostly depends on detection by professional medical devices at hospitals, while community hospitals and clinics with limited medical facilities do not have the corresponding detection and diagnosis capabilities. Therefore, they cannot effectively implement a rapid diagnosis and a preliminary diagnosis of cardiovascular and cerebrovascular diseases.

Therefore, a metadata-based diagnosis and management system for cardiovascular and cerebrovascular diseases is proposed.

### BRIEF SUMMARY OF THE INVENTION

In view of the disadvantages existing in the prior art, an objective of the present invention is to provide a metadata-based diagnosis and management system for cardiovascular and cerebrovascular diseases.

A technical problem to be resolved in the present invention is:
how to implement a rapid diagnosis and a preliminary diagnosis of cardiovascular and cerebrovascular diseases at places with limited medical facilities.

The objective of the present invention can be achieved by using the following technical solution.

A metadata-based diagnosis and management system for cardiovascular and cerebrovascular diseases includes a data collection module, a physical status monitoring module, a preliminary diagnosis module, an electrocardiogram comparison module, a model building module, a big data module, and a server, where the big data module is configured to obtain a large amount of medical and health data on cardiovascular and cerebrovascular diseases and send the data to the model building module, and the model building module is configured to build a disease diagnosis model with reference to the medical and health data on cardiovascular and cerebrovascular diseases and feedback the disease diagnosis model to the server for storage; the data collection module is configured to collect electrocardiogram data and physical status data of a person to be diagnosed and send the electrocardiogram data and physical status data to the server, and the server sends the electrocardiogram data to the electrocardiogram comparison module and the physical status data to the physical status monitoring module;
the electrocardiogram comparison module is configured to compare an electrocardiogram of cardiovascular and cerebrovascular systems of the person to be diagnosed to obtain an electrocardiogram deviation level and a corresponding electrocardiogram deviation coefficient and feedback the electrocardiogram deviation level and the electrocardiogram deviation coefficient to the server, and the server sends the electrocardiogram deviation level and the corresponding electrocardiogram deviation coefficient to the preliminary diagnosis module; the physical status monitoring module is configured to monitor a physical status of the person to be diagnosed to obtain a physical status deviation level and a corresponding physical status deviation coefficient and feedback the physical status deviation level and the physical status deviation coefficient to the server, and the server sends the physical status deviation level and the corresponding physical status deviation coefficient to the preliminary diagnosis module;
the preliminary diagnosis module is configured to make a preliminary diagnosis of cardiovascular and cerebrovascular diseases for the person to be diagnosed to generate a signal indicating a healthy body, a signal indicating reexamination, or a signal indicating a diagnosis, and feedback the signal to the server; if the server receives the signal indicating a healthy body, no operation is performed; if the server receives the signal indicating reexamination, physical conditions of the person to be diagnosed are monitored and diagnosed again; and if the server receives the signal indicating a disease, a disease query instruction is generated and then loaded into the data collection module; and
the data collection module retakes various physical feature values of the person to be diagnosed that are required for the disease diagnosis model, and sends the physical feature values to the server, and the server inputs the various physical feature values of the person to be diagnosed to the disease diagnosis model to obtain a diagnostic report of the person to be diagnosed by using the disease diagnosis model.

Further, a building process of the model building module specifically includes the following:
screening various physical feature values of patients with cardiovascular and cerebrovascular diseases in the medical and health data on cardiovascular and cerebrovascular diseases, where the various physical feature values constitute sample data of the cardiovascular and cerebrovascular diseases;
performing screening in the sample data again to obtain assessment factors of the cardiovascular and cerebrovascular diseases; and
building the disease diagnosis model for conditions of the cardiovascular and cerebrovascular diseases, based on the assessment factors of the cardiovascular and cerebrovascular diseases and by using a big data-based information analysis and mining algorithm.

Further, the electrocardiogram data includes a diagnostic electrocardiogram, a dynamic electrocardiogram, and an echocardiogram of the person to be diagnosed; and the physical status data includes a blood glucose concentration value, glycosylated hemoglobin, a glycosylated hemoglobin value, and a glycated serum protein value of the person to be diagnosed.

Further, a comparison process of the electrocardiogram comparison module specifically includes the following steps:
step 1: denoting the person to be diagnosed as u, where u=1, 2, . . . , and z, and z is a positive integer; and obtaining a diagnostic electrocardiogram corresponding to the person to be diagnosed;
step 2: obtaining an age, a gender, and a disease to be queried of the person to be diagnosed, and obtaining a corresponding preset electrocardiogram stored in the server based on the age, the gender, and the disease to be queried;
step 3: comparing the diagnostic electrocardiogram with the preset electrocardiogram to obtain an electrocardiogram deviation value XPu corresponding to the diagnostic electrocardiogram of the person to be diagnosed, where a comparison process specifically includes the following:
stacking the diagnostic electrocardiogram on the preset electrocardiogram;
calculating a crossing number JCu when the diagnostic electrocardiogram and the preset electrocardiogram are stacked;
obtaining a crossing region between the diagnostic electrocardiogram and the preset electrocardiogram based on the crossing number, and calculating an area of the crossing region to obtain a crossing area JMu; and
obtaining the electrocardiogram deviation value XPu corresponding to the diagnostic electrocardiogram of the person to be diagnosed through calculation by using a formula XPu=JCu×a1+JMu×a2, where both a1 and a2 are weight coefficients having fixed values, and values of both a1 and a2 are greater than zero; and
step 4: if XPu<X1, the electrocardiogram deviation level of the person to be diagnosed being a first electrocardiogram deviation level, and setting the corresponding electrocardiogram deviation coefficient;
if X1≤XPu<X2, the electrocardiogram deviation level of the person to be diagnosed being a second electrocardiogram deviation level, and setting the corresponding electrocardiogram deviation coefficient; and
if X2≤XPu, the electrocardiogram deviation level of the person to be diagnosed being a third electrocardiogram deviation level, and setting the corresponding electrocardiogram deviation coefficient, where both X1 and X2 are electrocardiogram deviation thresholds having fixed values, and X1<X2.

Further, the electrocardiogram deviation coefficient corresponding to the first electrocardiogram deviation level is less than the electrocardiogram deviation coefficient corresponding to the second electrocardiogram deviation level, and the electrocardiogram deviation coefficient corresponding to the second electrocardiogram deviation level is less than the electrocardiogram deviation coefficient corresponding to the third electrocardiogram deviation level.

Further, a monitoring process of the physical status monitoring module specifically includes the following steps:
step S1: obtaining the blood glucose concentration value, the glycosylated hemoglobin value, and the glycated serum protein value of the person to be diagnosed;
step S2: obtaining, according to the age, the gender, and the disease to be queried, corresponding ranges of blood glucose concentration values, glycosylated hemoglobin values, and glycated serum protein values that are stored in the server;
step S3: if the blood glucose concentration value, the glycosylated hemoglobin value, and the glycated serum protein value are all within the corresponding ranges, performing no operation; and
if any one of the blood glucose concentration value, the glycosylated hemoglobin value, and the glycated serum protein value falls outside the corresponding ranges, calculating deviation values of the blood glucose concentration value, the glycosylated hemoglobin value, and the glycated serum protein value relative to the corresponding ranges, and denoting the deviation values as XNCu, THCu, and TJCu;
step S4: obtaining a physical status deviation value TPu of the person to be diagnosed through calculation by using a formula TPu=XNCu×b1+THCu×b2+TJCu×b3, where b1, b2, and b3 are all weight coefficients having fixed values, and values of b1, b2, and b3 are all greater than zero; and
step S5: if TPu<Y1, the physical status deviation level of the person to be diagnosed being a first physical status deviation level, and setting the corresponding physical status deviation coefficient;
if Y1≤TPu<Y2, the physical status deviation level of the person to be diagnosed being a second physical status deviation level, and setting the corresponding physical status deviation coefficient; and
if Y2≤TPu, the physical status deviation level of the person to be diagnosed being a third physical status deviation level, and setting the corresponding physical status deviation coefficient, where both Y1 and Y2 are physical status deviation thresholds having fixed values, and Y1<Y2.

Further, the physical status deviation coefficient corresponding to the first physical status deviation level is less than the physical status deviation coefficient corresponding to the second physical status deviation level, and the physical status deviation coefficient corresponding to the second physical status deviation level is less than the physical status deviation coefficient corresponding to the third physical status deviation level.

Further, a preliminary diagnosis process of the preliminary diagnosis module specifically includes the following steps:
step SS1: denoting, as TPXu and XPXu respectively, the physical status deviation coefficient and the electrocardiogram deviation coefficient of the person to be diagnosed;
step SS2: obtaining a diagnostic value ZDuSPu of the person to be diagnosed through calculation by using a formula

$$ZDu = \frac{c 1}{c 2} \times TPXu + \frac{c 2}{c 3} \times XPXu ,$$

where c1, c2, and c3 are all proportionality coefficients having fixed values, and values of c1, c2, and c3 are all greater than zero;
step SS3: obtaining diagnostic ranges stored in the server, and comparing the diagnostic value of the person to be diagnosed with the diagnostic ranges to obtain a corresponding diagnostic range; and
step SS4: because different diagnostic ranges correspond to different preliminary diagnosis signals, obtaining a preliminary diagnosis signal of the person to be diagnosed according to the diagnostic range.

Further, the diagnostic range includes a first diagnostic range, a second diagnostic range, and a third diagnostic range, an upper limit of the first diagnostic range is less than a lower limit of the second diagnostic range, and an upper limit of the second diagnostic range is less than a lower limit of the third diagnostic range; and the first diagnostic range corresponds to the signal indicating a healthy body, the second diagnostic range corresponds to the signal indicating reexamination, and the third diagnostic range corresponds to the signal indicating a diagnosis.

Compared with the prior art, the beneficial effects of the present invention are as follows:

In the present invention, the model building module builds the disease diagnosis model, and feeds back the disease diagnosis model to the server for storage. In addition, the electrocardiogram comparison module compares an electrocardiogram of cardiovascular and cerebrovascular systems of the person to be diagnosed to obtain the electrocardiogram deviation level and the corresponding electrocardiogram deviation coefficient of the diagnostic electrocardiogram of the person to be diagnosed. Then, the physical status monitoring module monitors the physical status of the person to be diagnosed to obtain the physical status deviation level and the corresponding physical status deviation coefficient of the person to be diagnosed. Finally, the preliminary diagnosis module makes the preliminary diagnosis of cardiovascular and cerebrovascular diseases for the person to be diagnosed to generate the signal indicating a healthy body, the signal indicating reexamination, or the signal indicating a diagnosis, and feedback the signal to the server. When the server receives the signal indicating a diagnosis, the data collection module retakes the various physical feature values of the person to be diagnosed that are required for the disease diagnosis model and inputs the various physical feature values to the disease diagnosis model to obtain the diagnostic report of the person to be diagnosed by using the disease diagnosis model. The present invention can implement the rapid diagnosis and the preliminary diagnosis of the cardiovascular and cerebrovascular diseases at places with limited medical facilities.

### BRIEF DESCRIPTION OF THE DRAWINGS

To facilitate understanding of those skilled in the art, the present invention is further described with reference to the accompanying drawings.

FIG. 1 is a block diagram of an overall system according to the present invention.

### DETAILED DESCRIPTION OF THE INVENTION

The following clearly and completely describes the technical solutions of the present invention with reference to the embodiments. Apparently, the described embodiments are merely some rather than all of the embodiments of the present invention. All other embodiments obtained by those of ordinary skill in the art based on the embodiments of the present invention without creative efforts shall fall within the scope of protection of the present invention.

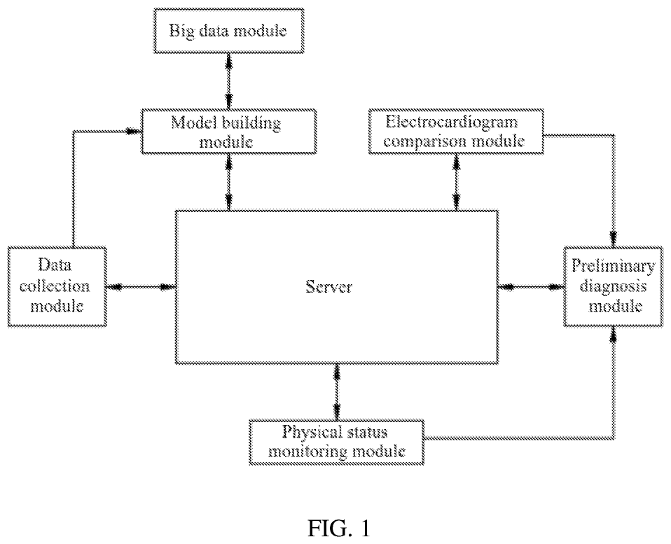

Refer to FIG. 1. A metadata-based diagnosis and management system for cardiovascular and cerebrovascular diseases includes a data collection module, a physical status monitoring module, a preliminary diagnosis module, an electrocardiogram comparison module, a model building module, a big data module, and a server.

The model building module is connected to the big data module. The big data module obtains a large amount of medical and health data on cardiovascular and cerebrovascular diseases by using Internet technologies, and sends the medical and health data on cardiovascular and cerebrovascular diseases to the model building module. The model building module is configured to build a disease diagnosis model with reference to the medical and health data on cardiovascular and cerebrovascular diseases, and a building process specifically includes the following:
screening various physical feature values of patients with cardiovascular and cerebrovascular diseases in the medical and health data on cardiovascular and cerebrovascular diseases, where the various physical feature values constitute sample data of the cardiovascular and cerebrovascular diseases; performing screening in the sample data again to obtain assessment factors of the cardiovascular and cerebrovascular diseases; and building the disease diagnosis model for conditions of the cardiovascular and cerebrovascular diseases, based on the assessment factors of the cardiovascular and cerebrovascular diseases and by using an advanced big data-based information analysis and mining algorithm, where in a specific implementation, the disease diagnosis model may be a Cox regression model and the like.

The model building module feeds back the disease diagnosis model to the server for storage.

The data collection module is configured to collect electrocardiogram data and physical status data of a person to be diagnosed and send the electrocardiogram data and the physical status data to the server.

It should be specifically noted that the electrocardiogram data includes a diagnostic electrocardiogram, a dynamic electrocardiogram, an echocardiogram, and the like of the person to be diagnosed. The physical status data includes a blood glucose concentration value, glycosylated hemoglobin, a glycosylated hemoglobin value, a glycated serum protein value, and the like of the person to be diagnosed.

In a specific implementation, both the electrocardiogram data and the physical status data may be accordingly collected according to requirements for the disease diagnosis, and are limited to such requirements. In addition, the data collection module is also a corresponding medical instrument for a cardiovascular and cerebrovascular disease diagnosis.

The server sends the electrocardiogram data to the electrocardiogram comparison module, and the server sends the physical status data to the physical status monitoring module.

The electrocardiogram comparison module is configured to compare an electrocardiogram of cardiovascular and cerebrovascular systems of the person to be diagnosed, and a comparison process specifically includes the following steps:
step 1: denoting the person to be diagnosed as u, where u=1, 2, . . . , and z, and z is a positive integer; and obtaining a diagnostic electrocardiogram corresponding to the person to be diagnosed;
step 2: obtaining an age, a gender, and a disease to be queried of the person to be diagnosed, and obtaining a corresponding preset electrocardiogram stored in the server based on the age, the gender, and the disease to be queried;
step 3: comparing the diagnostic electrocardiogram with the preset electrocardiogram to obtain an electrocardiogram deviation value XPu corresponding to the diagnostic electrocardiogram of the person to be diagnosed, where a comparison process specifically includes the following:
stacking the diagnostic electrocardiogram on the preset electrocardiogram;
calculating a crossing number JCu when the diagnostic electrocardiogram and the preset electrocardiogram are stacked;
obtaining a crossing region between the diagnostic electrocardiogram and the preset electrocardiogram based on the crossing number, and calculating an area of the crossing region to obtain a crossing area JMu; and
obtaining the electrocardiogram deviation value XPu corresponding to the diagnostic electrocardiogram of the person to be diagnosed through calculation by using a formula XPu=JCu×a1+JMu×a2, where both a1 and a2 are weight coefficients having fixed values, and values of both a1 and a2 are greater than zero; and
step 4: if XPu<X1, the electrocardiogram deviation level of the person to be diagnosed being a first electrocardiogram deviation level, and setting the corresponding electrocardiogram deviation coefficient;
if X1≤XPu<X2, the electrocardiogram deviation level of the person to be diagnosed being a second electrocardiogram deviation level, and setting the corresponding electrocardiogram deviation coefficient; and
if X2≤XPu, the electrocardiogram deviation level of the person to be diagnosed being a third electrocardiogram deviation level, and setting the corresponding electrocardiogram deviation coefficient, where both X1 and X2 are electrocardiogram deviation thresholds having fixed values, and X1<X2.

It should be specifically noted that the electrocardiogram deviation coefficient corresponding to the first electrocardiogram deviation level is less than the electrocardiogram deviation coefficient corresponding to the second electrocardiogram deviation level, and the electrocardiogram deviation coefficient corresponding to the second electrocardiogram deviation level is less than the electrocardiogram deviation coefficient corresponding to the third electrocardiogram deviation level.

In a specific implementation, the corresponding deviation values may be calculated by using not only the electrocardiogram but also the dynamic electrocardiogram and the echocardiogram. The deviation values may not only be a deviation value of the electrocardiogram but also values that are obtained through calculation by using deviation values of the electrocardiogram, the dynamic electrocardiogram, and the echocardiogram, and the weight coefficients. Corresponding data may be collected according to requirements for the disease diagnosis.

The electrocardiogram comparison module feeds back the electrocardiogram deviation level and the corresponding electrocardiogram deviation coefficient of the diagnostic electrocardiogram of the person to be diagnosed to the server, and the server sends the electrocardiogram deviation level and the corresponding electrocardiogram deviation coefficient of the diagnostic electrocardiogram of the person to be diagnosed to the preliminary diagnosis module.

The physical status monitoring module is configured to monitor a physical status of the person to be diagnosed, and a monitoring process specifically includes the following steps:
step S1: obtaining the blood glucose concentration value, the glycosylated hemoglobin value, and the glycated serum protein value of the person to be diagnosed;
step S2: obtaining, according to the age, the gender, and the disease to be queried, corresponding ranges of blood glucose concentration values, glycosylated hemoglobin values, and glycated serum protein values that are stored in the server;
step S3: if the blood glucose concentration value, the glycosylated hemoglobin value, and the glycated serum protein value are all within the corresponding ranges, performing no operation; and
if any one of the blood glucose concentration value, the glycosylated hemoglobin value, and the glycated serum protein value falls outside the corresponding ranges, calculating deviation values of the blood glucose concentration value, the glycosylated hemoglobin value, and the glycated serum protein value relative to the corresponding ranges, and denoting the deviation values as XNCu, THCu, and TJCu;
step S4: obtaining a physical status deviation value TPu of the person to be diagnosed through calculation by using a formula TPu=XNCu×b1+THCu×b2+TJCu×b3, where b1, b2, and b3 are all weight coefficients having fixed values, and values of b1, b2, and b3 are all greater than zero; and
step S5: if TPu<Y1, the physical status deviation level of the person to be diagnosed being a first physical status deviation level, and setting the corresponding physical status deviation coefficient;
if Y1≤TPu<Y2, the physical status deviation level of the person to be diagnosed being a second physical status deviation level, and setting the corresponding physical status deviation coefficient; and
if Y2≤TPu, the physical status deviation level of the person to be diagnosed being a third physical status deviation level, and setting the corresponding physical status deviation coefficient, where both Y1 and Y2 are physical status deviation thresholds having fixed values, and Y1<Y2.

It should be specifically noted that the physical status deviation coefficient corresponding to the first physical status deviation level is less than the physical status deviation coefficient corresponding to the second physical status deviation level, and the physical status deviation coefficient corresponding to the second physical status deviation level is less than the physical status deviation coefficient corresponding to the third physical status deviation level.

The physical status monitoring module feeds back the physical status deviation level and the corresponding physical status deviation coefficient of the person to be diagnosed to the server, and the server sends the physical status deviation level and the corresponding physical status deviation coefficient of the person to be diagnosed to the preliminary diagnosis module.

The preliminary diagnosis module is configured to make a preliminary diagnosis of cardiovascular and cerebrovascular diseases for the person to be diagnosed, and a preliminary diagnosis process specifically includes the following steps:
step SS1: denoting, as TPXu and XPXu respectively, the physical status deviation coefficient and the electrocardiogram deviation coefficient of the person to be diagnosed;
step SS2: obtaining a diagnostic value ZDuSPu of the person to be diagnosed through calculation by using a formula

$$ZDu = \frac{c 1}{c 2} \times TPXu + \frac{c 2}{c 3} \times XPXu ,$$

where c1, c2, and c3 are all proportionality coefficients having fixed values, and values of c1, c2, and c3 are all greater than zero, provided that in a specific implementation, values of the proportionality coefficients do not affect direct and inverse proportional relationships between parameters and result values;
step SS3: obtaining diagnostic ranges stored in the server, and comparing the diagnostic value of the person to be diagnosed with the diagnostic ranges to obtain a corresponding diagnostic range, where
the diagnostic range includes a first diagnostic range, a second diagnostic range, and a third diagnostic range, an upper limit of the first diagnostic range is less than a lower limit of the second diagnostic range, and an upper limit of the second diagnostic range is less than a lower limit of the third diagnostic range; and
step SS4: because different diagnostic ranges correspond to different preliminary diagnosis signals, obtaining a preliminary diagnosis signal of the person to be diagnosed according to the diagnostic range.

It should be specifically noted that the first diagnostic range corresponds to the signal indicating a healthy body, the second diagnostic range corresponds to the signal indicating reexamination, and the third diagnostic range corresponds to the signal indicating a diagnosis.

The preliminary diagnosis module feeds back the signal indicating a healthy body, the signal indicating reexamination, or the signal indicating a diagnosis to the server.

Specifically, if the server receives the signal indicating a healthy body, no operation is performed;
if the server receives the signal indicating reexamination, physical conditions of the person to be diagnosed are monitored and diagnosed again by using the physical status monitoring module, the electrocardiogram comparison module, and the preliminary diagnosis module; and
if the server receives the signal indicating a disease, a disease query instruction is generated and then loaded into the data collection module.

The data collection module retakes various physical feature values of the person to be diagnosed that are required for the disease diagnosis model, and sends the physical feature values to the server, and the server inputs the various physical feature values of the person to be diagnosed to the disease diagnosis model to obtain a diagnostic report of the person to be diagnosed by using the disease diagnosis model.

When the metadata-based diagnosis and management system for cardiovascular and cerebrovascular diseases works, the big data module obtains a large amount of medical and health data on cardiovascular and cerebrovascular diseases by using Internet technologies, and sends the medical and health data on cardiovascular and cerebrovascular diseases to the model building module. The model building module is configured to build a disease diagnosis model for conditions of the cardiovascular and cerebrovascular diseases with reference to the medical and health data on cardiovascular and cerebrovascular diseases, and the model building module feeds back the disease diagnosis module to the server for storage.

The data collection module collects the electrocardiogram data and the physical status data of the person to be diagnosed and sends the electrocardiogram data and the physical status data to the server, the server sends the electrocardiogram data to the electrocardiogram comparison module, and the server sends the physical status data to the physical status monitoring module.

The electrocardiogram comparison module compares the electrocardiogram of the cardiovascular and cerebrovascular systems of the person to be diagnosed, and denotes the person to be diagnosed as u. Then, the corresponding diagnostic electrocardiogram of the person to be diagnosed is obtained, and the corresponding preset electrocardiogram stored in the server is obtained based on the age, the gender, and the disease to be queried of the person to be diagnosed. The diagnostic electrocardiogram is compared with the preset electrocardiogram to obtain the electrocardiogram deviation value XPu of the diagnostic electrocardiogram corresponding to the person to be diagnosed. If XPu<X1, the electrocardiogram deviation level of the person to be diagnosed is the first electrocardiogram deviation level, and the corresponding electrocardiogram deviation coefficient is set. If X1≤XPu<X2, the electrocardiogram deviation level of the person to be diagnosed is the second electrocardiogram deviation level, and the corresponding electrocardiogram deviation coefficient is set. If X2≤XPu, the electrocardiogram deviation level of the person to be diagnosed is the third electrocardiogram deviation level, and the corresponding electrocardiogram deviation coefficient is set. The electrocardiogram comparison module feeds back the electrocardiogram deviation level and the corresponding electrocardiogram deviation coefficient of the diagnostic electrocardiogram of the person to be diagnosed to the server, and the server sends the electrocardiogram deviation level and the corresponding electrocardiogram deviation coefficient of the diagnostic electrocardiogram of the person to be diagnosed to the preliminary diagnosis module.

The physical status monitoring module monitors the physical status of the person to be diagnosed to obtain the blood glucose concentration value, the glycosylated hemoglobin value, and the glycated serum protein value of the person to be diagnosed. The corresponding ranges of the blood glucose concentration values, the glycosylated hemoglobin values, and the glycated serum protein values that are stored in the server are obtained according to the age, the gender, and the disease to be queried. If the blood glucose concentration value, the glycosylated hemoglobin value, and the glycated serum protein value are all within the corresponding ranges, no operation is performed. If any one of the blood glucose concentration value, the glycosylated hemoglobin value, and the glycated serum protein value falls outside the corresponding ranges, the deviation values of the blood glucose concentration value, the glycosylated hemoglobin value, and the glycated serum protein value relative to the corresponding ranges are calculated, and the deviation values are denoted as XNCu, THCu, and TJCu. The physical status deviation value TPu of the person to be diagnosed is obtained through calculation by using the formula TPu=XNCu×b1+THCu×b2+TJCu×b3. If TPu<Y1, the physical status deviation level of the person to be diagnosed is the first physical status deviation level, and the corresponding physical status deviation coefficient is set. If Y1≤TPu<Y2, the physical status deviation level of the person to be diagnosed is the second physical status deviation level, and the corresponding physical status deviation coefficient is set. If Y2≤TPu, the physical status deviation level of the person to be diagnosed is the third physical status deviation level, and the corresponding physical status deviation coefficient is set. The physical status monitoring module feeds back the physical status deviation level and the corresponding physical status deviation coefficient of the person to be diagnosed to the server, and the server sends the physical status deviation level and the corresponding physical status deviation coefficient of the person to be diagnosed to the preliminary diagnosis module.

The preliminary diagnosis module makes the preliminary diagnosis of cardiovascular and cerebrovascular diseases for the person to be diagnosed, and denotes, as TPXu and XPXu respectively, the physical status deviation coefficient and the electrocardiogram deviation coefficient of the person to be diagnosed. The diagnostic value ZDuSPu of the person to be diagnosed is obtained through calculation by using the formula

$$ZDu = \frac{c 1}{c 2} \times TPXu + \frac{c 2}{c 3} \times XPXu .$$

The diagnostic ranges stored in the server are obtained, and the diagnostic value of the person to be diagnosed is compared with the diagnostic ranges to obtain the corresponding diagnostic range. Because different diagnostic ranges correspond to different preliminary diagnosis signals, the preliminary diagnosis signal of the person to be diagnosed is obtained according to the diagnostic range. The preliminary diagnosis module feeds back the signal indicating a healthy body, the signal indicating reexamination, or the signal indicating a diagnosis to the server. If the server receives the signal indicating a healthy body, no operation is performed. If the server receives the signal indicating reexamination, the physical conditions of the person to be diagnosed are monitored and diagnosed again by using the physical status monitoring module, the electrocardiogram comparison module, and the preliminary diagnosis module. If the server receives the signal indicating a disease, the disease query instruction is generated and then loaded into the data collection module.

In addition, the data collection module retakes the various physical feature values of the person to be diagnosed that are required for the disease diagnosis model, and sends the physical feature values to the server, and the server inputs the various physical feature values of the person to be diagnosed to the disease diagnosis model to obtain a diagnostic report of the person to be diagnosed by using the disease diagnosis model.

Calculations of the foregoing formula are all performed by using dimensionless values. The formula is a formula that represents the most realistic situation by collecting a large amount of data for software simulation. The preset parameters in the formula are set by those skilled in the art according to an actual situation. Values of the weight coefficient and the proportionality coefficient are specific values obtained by quantifying each parameter for subsequent comparison. Values of the weight coefficients and the proportionality coefficient are set provided that the proportional relationship between the parameters and the values obtained by quantifying is not affected.

The preferred embodiments of the present invention disclosed above are only used to help illustrate the present invention. The preferred embodiments do not exhaust all the details, nor limit the present invention to only the specific embodiments described. Obviously, many modifications and changes can be made according to the content of this specification. In this specification, selection and specific descriptions of these embodiments are to better explain the principles and practical applications of the present invention, so that those skilled in the art can understand and use the present invention well. The present invention is only limited by the claims and their full scope and equivalents.

What is claimed is:

1. A metadata-based diagnosis and management system for cardiovascular and cerebrovascular diseases, comprising a data collection module, a physical status monitoring module, a preliminary diagnosis module, an electrocardiogram comparison module, a model building module, a big data module, and a server, wherein the big data module is configured to obtain a large amount of medical and health data on cardiovascular and cerebrovascular diseases and send the data to the model building module, and the model building module is configured to build a disease diagnosis model with reference to the medical and health data on cardiovascular and cerebrovascular diseases and feedback the disease diagnosis model to the server for storage; the data collection module is configured to collect electrocardiogram data and physical status data of a person to be diagnosed and send the electrocardiogram data and physical status data to the server, and the server sends the electrocardiogram data to the electrocardiogram comparison module and the physical status data to the physical status monitoring module;
the electrocardiogram comparison module is configured to compare an electrocardiogram of cardiovascular and cerebrovascular systems of the person to be diagnosed to obtain an electrocardiogram deviation level and a corresponding electrocardiogram deviation coefficient and feedback the electrocardiogram deviation level and the electrocardiogram deviation coefficient to the server, and the server sends the electrocardiogram deviation level and the corresponding electrocardiogram deviation coefficient to the preliminary diagnosis module; the physical status monitoring module is configured to monitor a physical status of the person to be diagnosed to obtain a physical status deviation level and a corresponding physical status deviation coefficient and feedback the physical status deviation level and the physical status deviation coefficient to the server, and the server sends the physical status deviation level and the corresponding physical status deviation coefficient to the preliminary diagnosis module;
the preliminary diagnosis module is configured to make a preliminary diagnosis of cardiovascular and cerebrovascular diseases for the person to be diagnosed to generate a signal indicating a healthy body, a signal indicating reexamination, or a signal indicating a diagnosis, and feedback the signal to the server; if the server receives the signal indicating a healthy body, no operation is performed; if the server receives the signal indicating reexamination, physical conditions of the person to be diagnosed are monitored and diagnosed again; and if the server receives the signal indicating a disease, a disease query instruction is generated and then loaded into the data collection module; and
the data collection module retakes various physical feature values of the person to be diagnosed that are required for the disease diagnosis model, and sends the physical feature values to the server, and the server inputs the various physical feature values of the person to be diagnosed to the disease diagnosis model to obtain a diagnostic report of the person to be diagnosed by using the disease diagnosis model.

2. The metadata-based diagnosis and management system for cardiovascular and cerebrovascular diseases according to claim 1, wherein a building process of the model building module specifically comprises the following:
screening various physical feature values of patients with cardiovascular and cerebrovascular diseases in the medical and health data on cardiovascular and cerebrovascular diseases, wherein the various physical feature values constitute sample data of the cardiovascular and cerebrovascular diseases;
performing screening in the sample data again to obtain assessment factors of the cardiovascular and cerebrovascular diseases; and
building the disease diagnosis model for conditions of the cardiovascular and cerebrovascular diseases, based on the assessment factors of the cardiovascular and cerebrovascular diseases and by using a big data-based information analysis and mining algorithm.

3. The metadata-based diagnosis and management system for cardiovascular and cerebrovascular diseases according to claim 1, wherein the electrocardiogram data comprises a diagnostic electrocardiogram, a dynamic electrocardiogram, and an echocardiogram of the person to be diagnosed; and
the physical status data comprises a blood glucose concentration value, glycosylated hemoglobin, a glycosylated hemoglobin value, and a glycated serum protein value of the person to be diagnosed.

4. The metadata-based diagnosis and management system for cardiovascular and cerebrovascular diseases according to claim 3, wherein a comparison process of the electrocardiogram comparison module specifically comprises the following steps:
step 1: denoting the person to be diagnosed as u, wherein u=1, 2, . . . , and z, and z is a positive integer; and obtaining the diagnostic electrocardiogram corresponding to the person to be diagnosed;
step 2: obtaining an age, a gender, and a disease to be queried of the person to be diagnosed, and obtaining a corresponding preset electrocardiogram stored in the server based on the age, the gender, and the disease to be queried;
step 3: comparing the diagnostic electrocardiogram with the preset electrocardiogram to obtain an electrocardiogram deviation value XPu corresponding to the diagnostic electrocardiogram of the person to be diagnosed, wherein the comparison process specifically comprises the following:
stacking the diagnostic electrocardiogram on the preset electrocardiogram;
calculating a crossing number JCu when the diagnostic electrocardiogram and the preset electrocardiogram are stacked;
obtaining a crossing region between the diagnostic electrocardiogram and the preset electrocardiogram based on the crossing number, and calculating an area of the crossing region to obtain a crossing area JMu; and
obtaining the electrocardiogram deviation value XPu corresponding to the diagnostic electrocardiogram of the person to be diagnosed through calculation by using a formula XPu=JCu×a1+JMu×a2, wherein both a1 and a2 are weight coefficients having fixed values, and values of both a1 and a2 are greater than zero; and
step 4: if XPu<X1, the electrocardiogram deviation level of the person to be diagnosed being a first electrocardiogram deviation level, and setting the corresponding electrocardiogram deviation coefficient;
if X1≤XPu<X2, the electrocardiogram deviation level of the person to be diagnosed being a second electrocardiogram deviation level, and setting the corresponding electrocardiogram deviation coefficient; and
if X2≤XPu, the electrocardiogram deviation level of the person to be diagnosed being a third electrocardiogram deviation level, and setting the corresponding electrocardiogram deviation coefficient, wherein both X1 and X2 are electrocardiogram deviation thresholds having fixed values, and X1<X2.

5. The metadata-based diagnosis and management system for cardiovascular and cerebrovascular diseases according to claim 4, wherein the electrocardiogram deviation coefficient corresponding to the first electrocardiogram deviation level is less than the electrocardiogram deviation coefficient corresponding to the second electrocardiogram deviation level, and the electrocardiogram deviation coefficient corresponding to the second electrocardiogram deviation level is less than the electrocardiogram deviation coefficient corresponding to the third electrocardiogram deviation level.

6. The metadata-based diagnosis and management system for cardiovascular and cerebrovascular diseases according to claim 4, wherein a monitoring process of the physical status monitoring module specifically comprises the following steps:
step S1: obtaining the blood glucose concentration value, the glycosylated hemoglobin value, and the glycated serum protein value of the person to be diagnosed;
step S2: obtaining, according to the age, the gender, and the disease to be queried, corresponding ranges of blood glucose concentration values, glycosylated hemoglobin values, and glycated serum protein values that are stored in the server;
step S3: if the blood glucose concentration value, the glycosylated hemoglobin value, and the glycated serum protein value are all within the corresponding ranges, performing no operation; and
if any one of the blood glucose concentration value, the glycosylated hemoglobin value, and the glycated serum protein value falls outside the corresponding ranges, calculating deviation values of the blood glucose concentration value, the glycosylated hemoglobin value, and the glycated serum protein value relative to the corresponding ranges, and denoting the deviation values as XNCu, THCu, and TJCu;
step S4: obtaining a physical status deviation value TPu of the person to be diagnosed through calculation by using a formula TPu=XNCu×b1+THCu×b2+TJCu×b3, wherein b1, b2, and b3 are all weight coefficients having fixed values, and values of b1, b2, and b3 are all greater than zero; and
step S5: if TPu<Y1, the physical status deviation level of the person to be diagnosed being a first physical status deviation level, and setting the corresponding physical status deviation coefficient;
if Y1≤TPu<Y2, the physical status deviation level of the person to be diagnosed being a second physical status deviation level, and setting the corresponding physical status deviation coefficient; and
if Y2≤TPu, the physical status deviation level of the person to be diagnosed being a third physical status deviation level, and setting the corresponding physical status deviation coefficient, wherein both Y1 and Y2 are physical status deviation thresholds having fixed values, and Y1<Y2.

7. The metadata-based diagnosis and management system for cardiovascular and cerebrovascular diseases according to claim 6, wherein the physical status deviation coefficient corresponding to the first physical status deviation level is less than the physical status deviation coefficient corresponding to the second physical status deviation level, and the physical status deviation coefficient corresponding to the second physical status deviation level is less than the physical status deviation coefficient corresponding to the third physical status deviation level.

8. The metadata-based diagnosis and management system for cardiovascular and cerebrovascular diseases according to claim 6, wherein a preliminary diagnosis process of the preliminary diagnosis module specifically comprises the following steps:
step SS1: denoting, as TPXu and XPXu respectively, the physical status deviation coefficient and the electrocardiogram deviation coefficient of the person to be diagnosed;
step SS2: obtaining a diagnostic value ZDuSPu of the person to be diagnosed through calculation by using a formula
$$ZDu = \frac{c 1}{c 2} \times TPXu + \frac{c 2}{c 3} \times XPXu ,$$
wherein c1, c2, and c3 are all proportionality coefficients having fixed values, and values of c1, c2, and c3 are all greater than zero;
step SS3: obtaining diagnostic ranges stored in the server, and comparing the diagnostic value of the person to be diagnosed with the diagnostic ranges to obtain a corresponding diagnostic range; and
step SS4: because different diagnostic ranges correspond to different preliminary diagnosis signals, obtaining a preliminary diagnosis signal of the person to be diagnosed according to the diagnostic range.

9. The metadata-based diagnosis and management system for cardiovascular and cerebrovascular diseases according to claim 8, wherein the diagnostic range comprises a first diagnostic range, a second diagnostic range, and a third diagnostic range, an upper limit of the first diagnostic range is less than a lower limit of the second diagnostic range, and an upper limit of the second diagnostic range is less than a lower limit of the third diagnostic range; and
the first diagnostic range corresponds to the signal indicating a healthy body, the second diagnostic range corresponds to the signal indicating reexamination, and the third diagnostic range corresponds to the signal indicating a diagnosis.
